(19) United States
(12) Patent Application Publication
(10) Pub. No.: US 2012/0123534 A1
(43) Pub. Date: May 17, 2012

(54) MODIFIED MONOVISION BY EXTENDING DEPTH OF FOCUS

(75) Inventors: Geunyoung YOON, Pittsford, NY (US); Leonard Zheleznyak, Rochester, NY (US); Ramkumar Sabesan, Rochester, NY (US)

(73) Assignee: University of Rochester, Rochester, NY (US)

(21) Appl. No.: 13/294,392

(22) Filed: Nov. 11, 2011

Related U.S. Application Data
(60) Provisional application No. 61/412,682, filed on Nov. 11, 2010.

(51) Int. Cl. A61F 2/16; A61B 18/20; G02C 7/02; G02C 7/04; A61B 3/00; A61B 3/02

(52) U.S. Cl. 623/6.11; 351/246; 351/222; 351/200; 351/159.01; 351/159.02; 606/4

(57) ABSTRACT

Depth-of-focus (DoF) is extended in a presbyopic patient by inducing different higher order aberrations, e.g. spherical aberration, to each of the two eyes. That method will result in improving binocular through-focus visual performance and outperform traditional monovision. The aberration can be induced in any suitable way, such as by an intraocular lens or a contact lens.

### REFERENCE TO RELATED APPLICATION

The present application claims the benefit of U.S. Provisional Patent Application No. 61/412,682, filed Nov. 11, 2010, whose disclosure is hereby incorporated by reference in its entirety into the present disclosure.

### FIELD OF THE INVENTION

The present invention is directed to treatment of presbyopia and more particularly to treatment of presbyopia binocularly by extending the depth of focus.

### DESCRIPTION OF RELATED ART

Presbyopia, the age-related loss of unassisted near vision, is a visual affliction which significantly affects anyone past the age of 60 years. A clinically well established approach towards correcting presbyopia is monovision (MV). In traditional monovision (TMV), the patient's dominant eye (DE) is refracted for distant vision and the non-dominant eye (NDE) for near vision, thus inducing anisometropia, different refractive error between the two eyes. Poor intermediate image quality and monocular suppression or inhibition of visual input can cause discomfort and reduced visual performance in presbyopic patients. That limitation is due to a large disparity in retinal image quality between the two eyes.

As noted above, monovision is a clinically well-established strategy for overcoming presbyopia. In traditional monovision, the dominant eye (DE) is refracted for distance vision, and the non-dominant eye (NDE) is refracted for near vision [1-3]. Monovision can be implemented with contact lenses [4-6], corneal refractive surgery [7, 8], or monofocal intraocular lenses following crystalline lens removal in cataract surgery [3, 9-11]. This approach to correcting presbyopia can improve distance corrected near vision, however, each eye is monofocal and remains presbyopic [2]. Therefore, the typical monovision patient has a relatively short monocular dynamic range of through-focus vision.

The power difference between the two eyes, or anisometropia, is the foundation of improving near vision in monovison for presbyopia. However, there is controversy in the literature over the most favorable amount of anisometropia. The optimal power difference between the two eyes, or anisometropia, has been reported to vary between 1.0 and 2.0 diopters (D), depending on the need for the patient's lifestyle or occupation [2, 12, 13]. It has been shown that anisometropia greater than 1.0 diopter is effective for improving distance-corrected near visual acuity [12, 13]. However, anisometropia greater than 2.0 D can significantly reduce intermediate visual performance and stereo acuity [2, 12]. The lack of a consensus for the optimal degree of anisometropia is likely caused by previous studies' inconsistencies in parameters which strongly influence through-focus retinal image quality, namely pupil size and higher order aberrations. For example, a subject with small pupils (and thus larger depth of focus, due to diffraction) may be able to tolerate a larger degree anisometropia, as compared to a subject with relatively large pupils.

A hallmark measure of binocular visual performance is binocular summation, the phenomenon in which binocular contrast sensitivity outperforms monocular contrast sensitivity, as demonstrated by Campbell and Green in 1965. In their experiment, diffraction-limited viewing (through 2.8mm pupils) with no anisometropia yielded a binocular summation factor (ratio of binocular to monocular contrast sensitivity) of roughly 41% [14]. However, binocular summation is known to decrease as the interocular diversity in retinal image quality increases. Pardhan and Gilchrist found that binocular summation at distance decreased as the magnitude of anisometropia in monovision increased. Interestingly, they found that when anisometropia is between 2.0 and 2.5 D, contrast sensitivity at 6 cycles per degree is poorer with binocular viewing than monocular viewing, i.e. the binocular summation factor was below unity [15]. Disparity in retinal image quality between the two eyes due to ocular differences in higher order aberrations has also been shown to decrease binocular summation [16, 17]. Therefore, it is important to minimize the difference in interocular retinal image quality at different through-focus positions to maintain binocular summation.

Bracketed numbers refer to the following references:

1. Jain, S., I. Arora, and D. T. Azar, Success of monovision in presbyopes: Review of the literature and potential applications to refractive surgery*. Survey of ophthalmology, 1996. 40(6): p. 491-499.

2. Johannsdottir, K. R. and L. B. Stelmach, Monovision: a review of the scientific literature. Optometry and vision science: official publication of the American Academy of Optometry, 2001. 78(9): p. 646.

3. Evans, B. J. W., Monovision: a review. Ophthalmic and Physiological Optics, 2007. 27(5): p. 417-439.

4. Collins, M. J. and A. S. Bruce, Factors influencing performance with monovision.

Journal of The British Contact Lens Association, 1994. 17(3): p. 83-89.

5. Fisher, K., Presbyopic visual performance with modified monovision using multifocal soft contact lenses. International Contact Lens Clinic, 1997. 24(3): p. 91-100.

6. McGill, E. and P. Erickson, Stereopsis in presbyopes wearing monovision and simultaneous vision bifocal contact lenses. American journal of optometry and physiological optics, 1988. 65(8): p. 619.

7. Goldberg, D. B., Laser in situ keratomileusis monovision 1. Journal of Cataract & Refractive Surgery, 2001. 27(9): p. 1449-1455.

8. Braun, E. H. P., J. Lee, and R. F. Steinert, Monovision in LASIK. Ophthalmology, 2008. 115(7): p. 1196-1202.

9. Greenbaum, S., Monovision pseudophakia. Journal of Cataract & Refractive Surgery, 2002. 28(8): p. 1439-1443.

10. Finkelman, Y. M., J. Q. Ng, and G. D. Barrett, Patient satisfaction and visual function after pseudophakic monovision. Journal of Cataract & Refractive Surgery, 2009. 35(6): p. 998-1002.

11. Ito, M., et al., Assessment of visual performance in pseudophakic monovision.

Journal of Cataract & Refractive Surgery, 2009. 35(4): p. 710-714.

12. Hayashi, K., et al., Optimal Amount of Anisometropia for Pseudophakic Monovision. Journal of refractive surgery (Thorofare, N.J.: 1995), 2010: p. 1.

13. Legras, R., et al., Effect of induced anisometropia on binocular through-focus contrast sensitivity. Optometry & Vision Science, 2001. 78(7): p. 503.

14. Campbell, F. W. and D. G. Green, Monocular versus binocular visual acuity. Nature, 1965. 208: p. 191-192.

15. Pardhan, S. and J. Gilchristt, The effect of monocular defocus on binocular contrast sensitivity. Ophthalmic and Physiological Optics, 1990. 10(1): p. 33-36.

16. JimEnez, J. E. R., et al., Interocular differences in higher-order aberrations on binocular visual performance. Optometry & Vision Science, 2008. 85(3): p. 174.

17. Jimenez, J. R., et al., Impact of interocular differences in corneal asphericity on binocular summation. American Journal of Ophthalmology, 2003. 135(3): p. 279-284.

18. Fernandez, E. J., P. M. Prieto, and P. Artal, Adaptive optics binocular visual simulator to study stereopsis in the presence of aberrations. JOSA A, 2010. 27(11): p. A48-A55.

19. Porac, C. and S. Coren, The dominant eye. Psychological Bulletin, 1976. 83(5): p.

880.

20. Walls, G. L., A theory of ocular dominance. Archives of Ophthalmology, 1951. 45(4): p. 387.

21. Handa, T., et al., Effects of ocular dominance on binocular summation after monocular reading adds. Journal of Cataract & Refractive Surgery, 2005. 31(8): p. 1588-1592.

22. Collins, M., A. Bruce, and B. Thompson, Adaptation to monovision. International Contact Lens Clinic, 1994. 21(11-12): p. 218-224.

23. Lovasik, J. V. and M. Szymkiw, Effects of aniseikonia, anisometropia, accommodation, retinal illuminance, and pupil size on stereopsis. Investigative ophthalmology & visual science, 1985. 26(5): p. 741.

### SUMMARY OF THE INVENTION

It is therefore an object of the invention to enhance through-focus binocular visual performance in presbyopia by manipulating the aberration of each of the two eyes, which reduces the disparity in optical quality.

To achieve the above and other objects, the present invention extends the depth-of-focus (DoF) by inducing different higher order aberrations, e.g. spherical aberration, to each of the two eyes. That method will result in improving binocular through-focus visual performance and outperform traditional monovision. The aberration can be induced in any suitable way, such as by laser refractive surgery as well as other potential surgical methods for refractive error correction, an intraocular lens, a contact lens or any other forms of ophthalmic methods. An adaptive optics (AO) vision simulator can be used in the laboratory and in fact has been used to test the concept, but for practical applications, a surgical procedure or an ophthalmic lens can be suitably formed to provide the required aberrations.

The present invention provides for decreasing through-focus interocular optical diversity to extend the depth of focus of the non-dominant eye with spherical aberration. The present invention allows counterbalancing the detrimental aspects of anisometropia (diversity in retinal image quality) by extending the depth of focus of the non-dominant eye with spherical aberration to improve through-focus visual performance and binocular summation.

Binocular adaptive optics (AO) is a powerful tool for vision testing [18]. It allows for the correction of all aberrations (lower and higher order) of both eyes simultaneously, after which higher-order aberrations were induced. Binocular AO can be used in the present invention, as will be explained in detail below.

Spherical aberration is one of many potential examples to increase DoF. Basically, any methods to increase DoF can be used in the present invention.

The aberration in the present invention can be applied to only one eye or both eyes. When applying it to both eyes, each of the two eyes can have the same aberration or different aberrations depending on the patient's needs or preferences.

Any suitable surgical procedures can be used in the present invention.

### BRIEF DESCRIPTION OF THE DRAWINGS

A preferred embodiment of the present invention will be set forth in detail with reference to the drawings, in which:

FIG. 1 is a flow chart showing the method of the preferred embodiment;

FIG. 2 is a block diagram of a system on which the method of FIG. 1 can be implemented;

FIGS. 3A and 3B show optics that can be used in the preferred embodiment;

FIGS. 4A-4E show plots of theoretical monocular image quality;

FIGS. 4F-4J show plots of theoretical binocular image quality;

FIGS. 5A-5E show experimental results for a first subject;

FIGS. 5F-5J show experimental results for a second subject;

FIG. 6 shows a comparison of theoretical and experimental results;

FIG. 7 is a graph showing through-focus binocular visual acuity for traditional and modified monovision conditions;

FIG. 8 is a graph showing binocular depth of focus for traditional and modified monovision conditions, in which the depth of focus threshold=20/30 visual acuity (p<0.05);

FIGS. 9A-9C are graphs showing through-focus monocular and binocular contrast sensitivity (at 10 cpd) for traditional (FIGS. 9A) and modified (FIGS. 9B and 9C) monovision conditions; and

FIGS. 10A-10D are graphs showing through-focus binocular summation factor for traditional (FIG. 10A) and modified (FIGS. 10B and 10C) monovision conditions for three individual subjects and their average (FIG. 10D), in which error bars signify one standard deviation.

### DETAILED DESCRIPTION OF THE PREFERRED EMBODIMENT

A preferred embodiment of the invention will be set forth below with reference to the drawings.

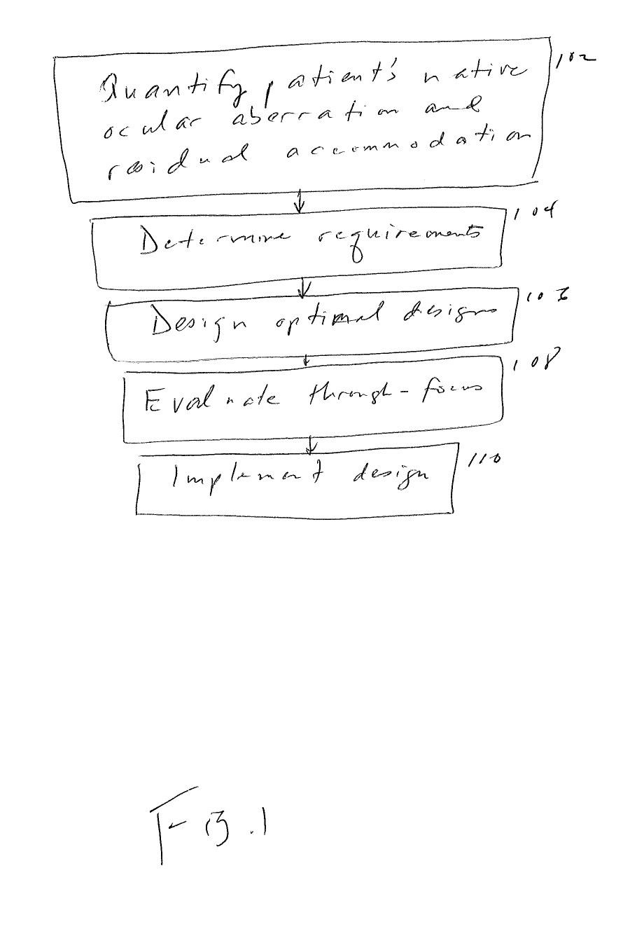

FIG. 1 is a flow chart showing the method of carrying out the preferred embodiment.

The method involves the following steps:

Step 102: Quantifying (or evaluating) the patient's native ocular aberration and residual accommodation;

Step 104: Determining the patient's requirements, i.e., a range of depth of focus and relative importance of distance vs intermediate vs near vision, which will determine the required amount of anisometropia;

Step 106: Designing optimal designs of induced aberration for each of the two eyes that take the factors above into account;

Step 108 Evaluating through-focus visual performance using AO vision simulator; and

Step 110: Implementing the design onto actual ophthalmic devices, e.g., refractive surgery, contact lens or IOL.

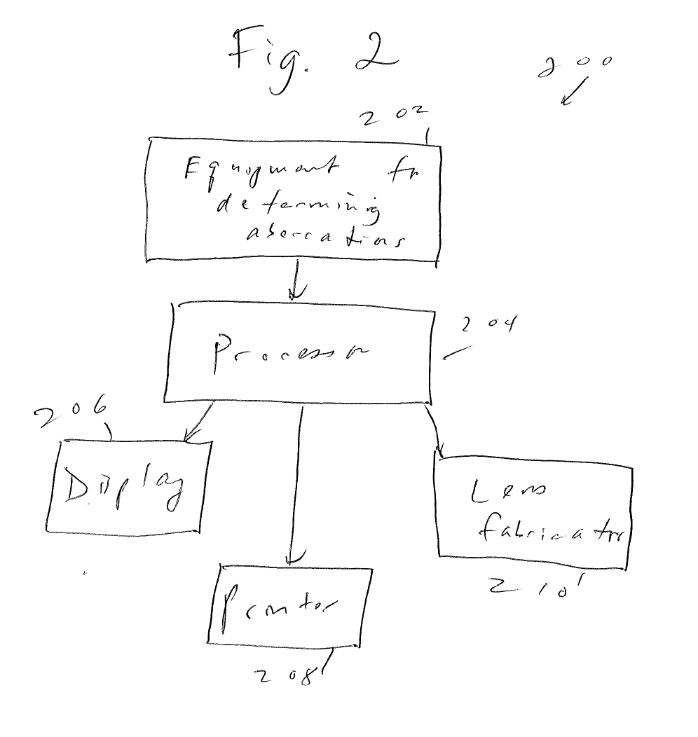

The method of FIG. 1 can be implemented on the system 200 of FIG. 2. Equipment 202 for determining the existing aberrations of the patient's eyes outputs that information to a suitably programmed processor 204, which carries out steps 102 and 104 and then outputs the requirements for the optics to at least one of a display 206, a printer 208, and a lens fabricator 210.

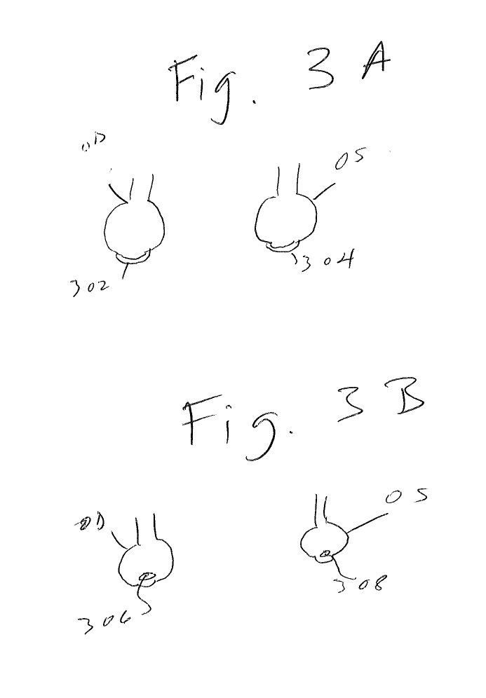

FIGS. 3A and 3B show different kinds of optics, which are different for the left eye OS and the right eye OD. The optics can be contact lenses 302, 304, intraocular lenses 306, 308, or any other suitable optics.

The concept was tested with two presbyopic subjects (age 42 and 58 years). High-contrast binocular visual acuity (Tumbling E) was measured with a binocular adaptive optics system. The through-focus values used were 0.00, 0.75, 1.50, 2.25, 3.00 diopters.

The subjects' pupils were dilated and residual accommodation paralyzed with tropicamide (1.0%) to form 6mm pupils. Spherical aberration was used to extend the depth of focus of the non-dominant eye. Binocular through-focus visual acuity was compared to the theoretical model based on the quadratic summation model applied to the visual Strehl ratio (VSOTF). The quadratic summation model is C=\sqrt{(C_{DE})²+(C_{NDE})²}, where the subscripts DE and NDE refer to the dominant and non-dominant eyes, respectively.

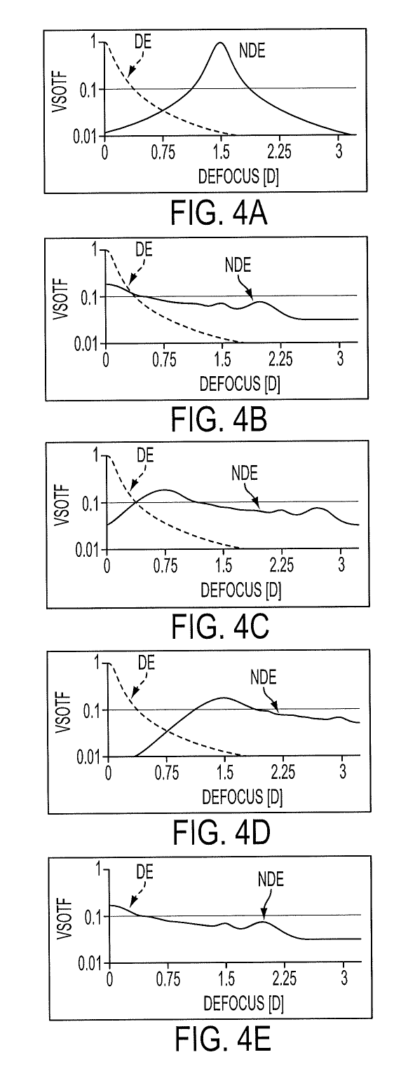

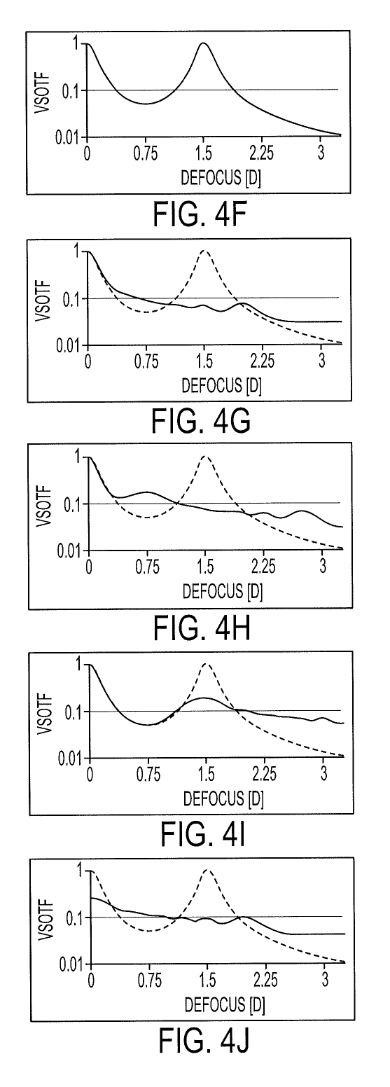

FIGS. 4A-4E show plots of the theoretical monocular image quality for the following five conditions, respectively:

TABLE I
The five conditions

| Condition | Dominant eye | Non-dominant eye |
|---|---|---|
| 1 | Aberration-free at 0 D | Aberration-free at 1.5 D |
| 2 | Aberration-free at 0 D | SA = +0.5 μm at 0 D |
| 3 | Aberration-free at 0 D | SA = +0.5 μm at 0.75 D |
| 4 | Aberration-free at 0 D | SA = +0.5 μm at 1.5 D |
| 5 | SA = +0.5 μm at 0 D | SA = +0.5 μm at 0 D |

FIGS. 4F-4J show plots of the theoretical prediction of the binocular visual performance for the same five conditions, respectively.

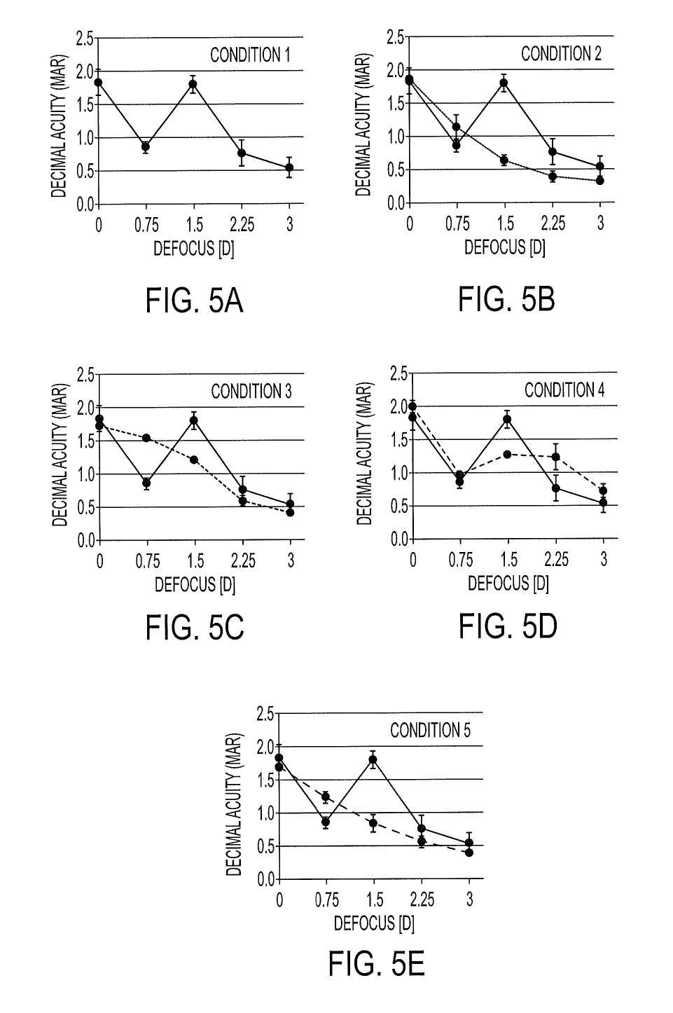

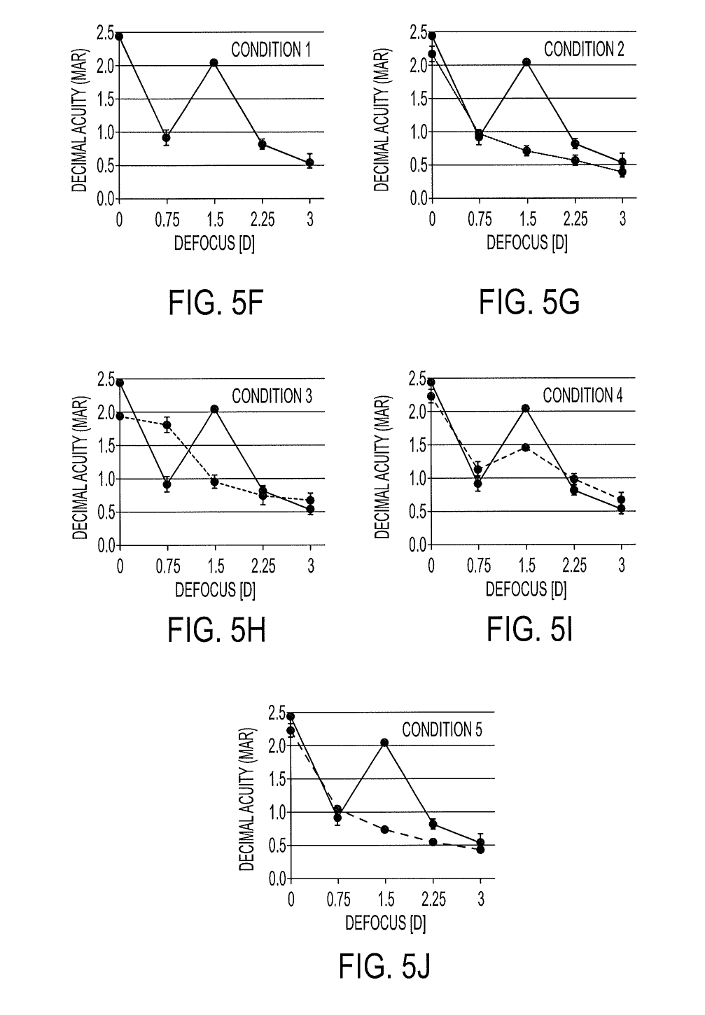

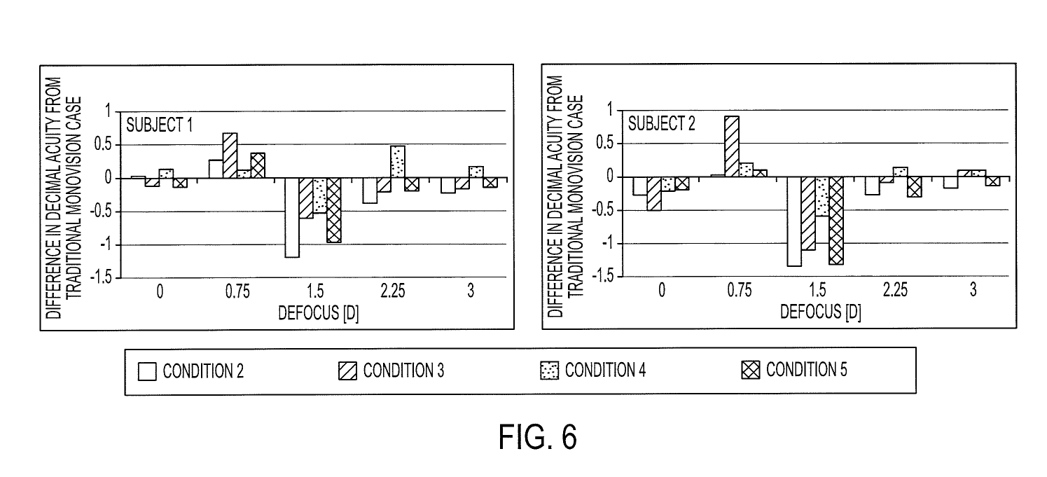

Experimental results for the five conditions are shown for the first subject in FIGS. 5A-5E and for the second subject in FIGS. 5F-5J. TMV and the present embodiment are compared in FIG. 6. As shown, conditions 2-5 improve visual performance at 0.75 D but reduce visual performance at 1.5 D, while condition 4 improves visual performance (2.25-3 D).

Accordingly, it will be seen that the quadratic summation model of binocular visual performance is a predictor of binocular through-focus visual acuity. Modified monovision using monocularly extended depth-of-focus will have significant potential in enhancing through-focus presbyopic visual performance.

Further experimental results will now be disclosed. Seven normal emmetropic subjects were recruited for this study (age: 34+/−11 years). All subjects' pupil were dilated and accommodation paralyzed with cyclopentalate hydrochloride (1%).

The binocular AO vision simulator consisted of two identical monocular AO systems, one allocated for each eye. Each monocular AO system was comprised of a custom-made Shack-Hartmann wavefront sensor, a large stroke deformable mirror (Imagine Eyes, Mirao-52), a Badal optometer, an artificial pupil and a visual stimulus display. The deformable mirror was used in closed-loop to manipulate the subjects' wavefront aberrations in real-time. The wavefront sensor laser beacon was produced by a super-luminescent diode with center wavelength of 840 nm and a bandwidth of 40 nm. Badal optometers were used to control object distance for through-focus vision testing. Aberration manipulation with AO was performed over a 6.0 mm pupil, however vision testing occurred in white light with the subject viewing through a 4.0 mm artificial pupil. During binocular vision testing, subjects reported fusion. A dental-impression bite bar mount was used to stabilize head movements. The eyes' pupils were monitored continuously with a camera focused at the pupil planes.

The dominant eye in all subjects was determined with a sighting test known as the Porta Test [19]. The sighting test consisted of the subject binocularly viewing an object directly in front of them and subsequently occluding one eye at a time. The dominant eye was determined to be the eye which produced lesser “jump” when the contralateral was occluded [20]. All monovision conditions in this investigation kept the dominant eye at 0 D, and fully corrected to be aberration-free. Also, all monovision conditions kept the non-dominant eye at 1.5 D of anisometropia under three higher-order aberration conditions: (1) traditional monovision with the non-dominant eye having zero higher-order aberrations, (2) modified monovision with the non-dominant eye having +0.2 mm of spherical aberration and (3) modified monovision with the non-dominant eye having −0.2 mm of spherical aberration.

To fix the non-dominant eye at 1.5 D in the presence of spherical aberration, additional defocus had to be added to the system. A Matlab simulation was performed to determine the required defocus shift for optimizing the non-dominant eye's retinal image quality at 1.5 D in the presence of positive and negative spherical aberration. Table II summarizes the results of the simulation.

TABLE II
Results of Matlab Simulation

| | DE | NDE | Defocus required to |
|---|---|---|---|
| | defocus | SA | bring RIQ peak to |
| | [D] | [um] | 1.5 D [D] |
| Traditional | 0 | 0 | 1.5 |
| Monovision | | | |
| Modified | 0 | +0.2 | 2.15 |
| Monovision (0, +) | | | |
| Modified | 0 | −0.2 | 0.85 |
| Monovision (0, −) | | | |

Visual performance was assessed both binocularly and monocularly with high-contrast visual acuity and contrast sensitivity. During monocular measurements, an eyepatch occluded the inactive eye.

High-contrast visual acuity (VA) was measured with a black tumbling letter E on a white background. Measurements were obtained through-focus in 0.5 D increments from far (0 D) to near (until VA<0.3 logMAR). The 0 D far point was determined monocularly for both eyes. To establish the 0 D point, the subject viewed a 20/40 letter E through a 4.0 mm pupil during AO aberration correction (over a 6.0 mm pupil). Subjective best-focus followed by adjusting the power in the system with the Badal optometer.

Monocular VA was measured in the dominant eye only. Monocular VA was measured with full aberration-correction with three different magnitudes of spherical aberration: 0 μm, −0.2 μM and +0.2 μm, through focus from 0-3.5 D. Binocular VA was measured for the three monovision conditions. Depth of focus was defined as the dioptric range where VA was better than 0.3 logMAR.

Contrast sensitivity was measured monocularly (both eyes) and binocularly with Gabor stimuli at 10 cycles per degree (cpd). The dominant eye was fully-corrected and measured through-focus (0-3 D). The non-dominant eye was measured through-focus (0-3 D) with 0, +0.2 and −0.2 μm of spherical aberration while optimized at 1.5 D. Binocular summation was defined as the ratio of the binocular contrast sensitivity to the monocular contrast sensitivity of the better eye.

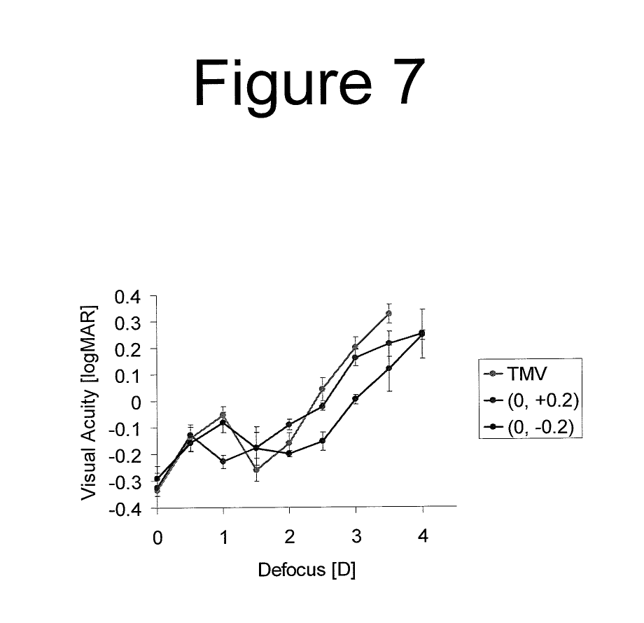

FIG. 7 summarizes the through-focus visual acuity measurements for the three monovision conditions (traditional monovision, with positive SA in the non-dominant eye, and with negative SA in the dominant eye). VA is plotted in units of the logarithm of the minimum angle of resolution (logMAR) as a function of defocus in units of diopters (D), where positive diopters indicate near objects. The error bars indicate the standard deviation from three subjects.

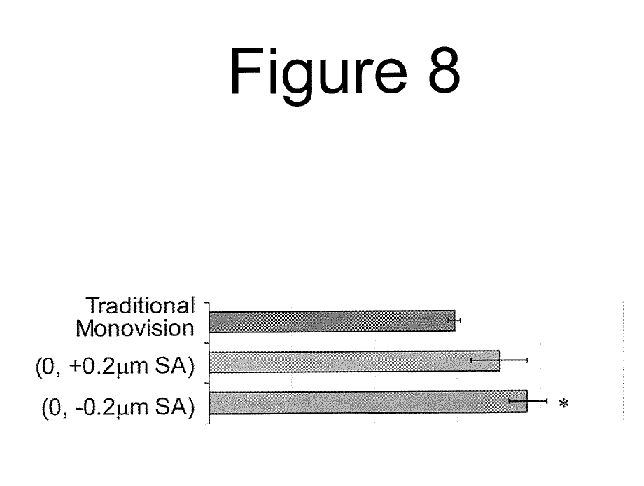

Modifying monovision by inducing SA in the non-dominant did not affect distance VA (at 0 D), which was better than −0.2 logMAR for the three monovision conditions. There was a significant improvement in binocular VA at 1 D with the case of modified monovision with positive SA in the non-dominant eye over traditional monovision and modified monovision with negative SA in the non-dominant eye. At the anisometropic point of 1.5 D, inducing spherical aberration in the non-dominant eye did not have a significant impact on VA, as compared to the traditional monovision condition. Both cases of modified monovision improved near VA (beyond 2 D) and thus extended binocular depth of focus, as seen in FIG. 8.

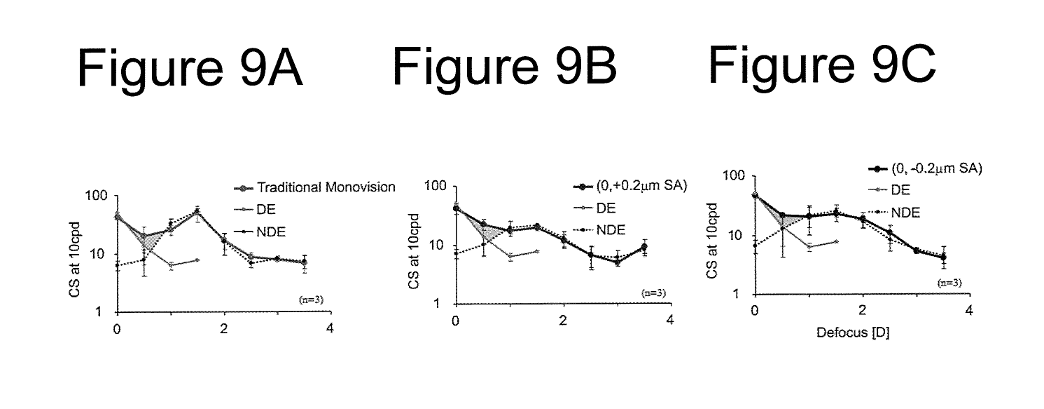

FIGS. 9A-9C summarize the through-focus monocular and binocular contrast sensitivity measurements for traditional (FIG. 9A) and the modified (FIGS. 9B and 9C) monovision conditions. As expected, the DE contrast sensitivity peaks at 0 D and quickly degrades with defocus. Similarly, in the traditional monovision condition, the NDE contrast sensitivity peaks at the anisometropic point of 1.5 D. As seen in FIGS. 9B and 9C, with positive and negative spherical aberration induced in the NDE, the peak contrast sensitivity remains the anisometropic point (1.5 D); however, it is reduced. The binocular contrast sensitivity trend follows the monocular contrast sensitivity through-focus closely except at the 0.5 D position, where binocular CS is superior to monocular CS, indicating binocular summation. The highlighted gold area illustrates the region of binocular summation.

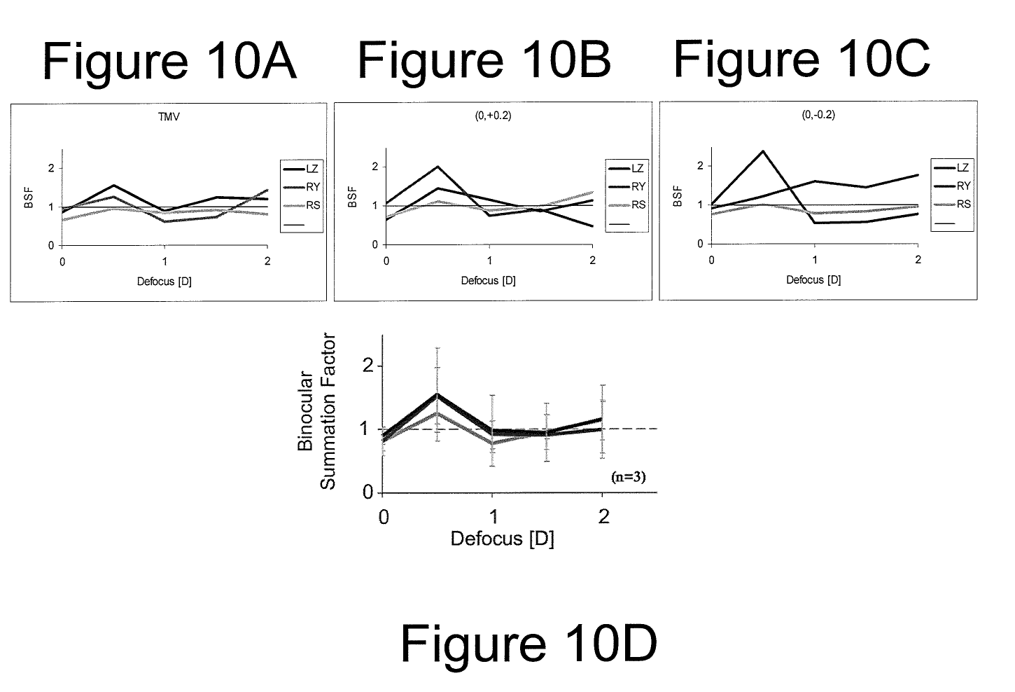

FIGS. 10A-10D summarize through-focus binocular summation between 0 and 2 D for the three subjects and their average. The error bars in FIG. 10D, which shows an average of the traditional (FIG. 10A) and modified (FIGS. 10B and 10C) binocular summation factors, signify the standard deviation among the through-focus binocular summation factor of the three subjects. At distance (0 D), the binocular summation factor is close to unity for all three monovision conditions. The average binocular summation increases at the 0.5 D position for traditional monovision to 1.25. For modified monovision with positive and negative SA induced in the NDE, the average binocular summation factor at 0.5 D was 1.53 and 1.55, respectively. Beyond 0.5 D, the average binocular summation factor for traditional and modified monovision conditions was approximately unity. However, on an individual basis, subjects LZ and RY demonstrated a benefit in binocular summation due to modifying monovision. Subject RS did not exhibit binocular summation for the majority of measured through-focus positions.

The binocular AO vision simulator is a powerful tool for the simulation of binocular presbyopic corrections. Binocular AO opens the doors to the investigation of presbyopic corrections which utilize the two ocular channels of the left and right eye independently. Subjects non-invasively experienced three monovision strategies: traditional monovision with aberration-free optical quality (FIGS. 9A and 10A), and modified monovision with positive (FIGS. 9B and 10B) and negative (FIGS. 9C and 10C) spherical aberration induced in the non-dominant eye.

The present disclosure reports the through-focus visual acuity measured under three varieties of monovision. Distance visual acuity among all three monovision conditions, tradition and modified with positive and negative spherical aberration induced in the NDE, was reasonably consistent of −0.33, −0.33 and −0.29 logMAR, respectively. Similarly, at 0.5 D all three monovision conditions were alike (−0.14, −0.13 and −0.16 logMAR, respectively).

However, at 1.0 D, the visual acuity for the monovision conditions diverged. Traditional monovision and modified monovision with negative spherical aberration had visual acuity of −0.05 and −0.08 logMAR, respectively. However, the positive spherical aberration induced in the NDE in monovision significantly improved visual acuity at 1.0 D to −0.23 logMAR. The NDE across the monovision conditions was optimized at 1.5 D. For traditional monovision, visual acuity at 1.5 D was −0.26 logMAR. Modified monovision with positive and negative spherical aberration induced in the NDE, reduced the visual acuity at 1.5 D by nearly one line, to −0.18 and −0.18 logMAR, respectively. Beyond 1.5 D, traditional monovision experienced the most rapid degradation in visual acuity. Inducing both positive and negative spherical aberration in the NDE extended binocular depth of focus in the near, however negative spherical aberration was more effective. Interestingly, positive and negative values of spherical aberration in the NDE had different effects on binocular visual acuity. Modified monovision with positive spherical aberration in the NDE yielded a benefit over rational monovision at the 1.0 D through-focus position, in the far-direction from the NDE's anisometropic point (defocus <1.5 D). Conversely, negative spherical aberration in the NDE yielded a benefit in visual acuity in the near-direction (defocus >1.5 D).

Traditional monovision, with aberration-free optical quality in both eyes, led to a depth of focus of 3.0 D. The binocular depth of focus improved by modifying monovision with positive and negative spherical aberration induction in the NDE (3.5 and 3.8 D, respectively). Therefore, correcting spherical aberration from the presbyopic eye (with aspheric intraocular lenses, for example) may not be an ideal approach for the prebyope seeking a spectacle-free lifestyle with monovision.

Pardhan and Gilchrist [15] illustrated the reduction in binocular summation as anisometropia was induced in the NDE. They found that for 0 D anisometropia, ratio of binocular to monocular contrast sensitivity was ˜40%, similar to the findings of Campbell and Green [14]. However as the anisometropia was increased, summation degraded, reaching unity (no summation) at 1.5 D anisometropia, beyond which summation was <1, indicating binocular inhibition. The results presented in this study also show distance binocular summation to be near unity for 1.5 D of anismetropia for traditional and modified monovision conditions, albeit a different spatial frequency was measured. Subject RS's lack of binocular summation in monovision may be due to strong ocular dominance, indicating a poor candidate for monovision. At 0.5 D, binocular summation >1 was observed for subjects LZ and RY, for all monovision conditions. Modifying monovision with spherical aberration in the NDE further improved through-focus binocular summation for subject LZ and RY. Modified monovision with negative, rather than positive, spherical aberration provided a larger benefit in through-focus binocular summation for LZ and RY. The inter-subject variability in the magnitude of binocular summation may be in part due to each subjects' strength of ocular dominance. Handa et al have shown that people with strong ocular dominance require the non-dominant eye to be allocated for near vision in order to exhibit binocular summation in monovision [21]. It is possible that the sighting test used in this study did not accurately determine the ocular dominance of subject RS, which may explain this subject's lack of summation.

A limitation of the study disclosed above is that the experimental protocol called for the use cyclopentalate to dilate the pupils and arrest accommodation. In natural viewing conditions, accommodative effort for viewing near objects is accompanied by pupil miosis, thereby extending ocular depth of field. Therefore, the lack of miosis in this study may have resulted in underestimated visual performance values for near objects.

Expansions on the invention can include the role of neural adaptation to traditional monovision and modified monovision. Collins et al. found that monovision wearers subjectively observed an improvement in some aspects of visual performance, such as walking confidence and hand-eye coordination [22]. Interestingly however, objective measures of visual performance such as high and low contrast visual acuity, stereoacuity and blur suppression showed no improvement as a function of time. By the nature of this investigation, subjects did not have the chance to wear the traditional and modified monovision corrections outside of the binocular AO vision simulator, i.e. for their daily lives. Therefore, they may have not had the opportune environment to facilitate neural adaption. It may be helpful in a neural adaptation study of modified monovision to use customized contact or scleral lenses to impart the correction to allow for experience with the correction during the subjects' daily lives.

Depth perception, a key component to binocular vision, was not examined in this study. It will be interesting to investigate the impact of monocularly extended depth of focus in monovision on stereoacuity. It is well-known that anisometropia in monovision leads to a reduction in stereoacuity [4, 6, 12, 23]. However, by extending the depth of focus of one or both eyes in monovision improves through focus retinal image quality, and therefore may improve stereoacuity in modified monovision [5].

While a preferred embodiment has been set forth in detail above, those skilled in the art who have reviewed the present disclosure will readily appreciate that other embodiments can be realized within the scope of the invention. For example, numerical values are illustrative rather than limiting, as are disclosures of particular types of lenses and particular substances. Therefore, the present invention should be construed as limited only by the appended claims.

I claim:

1. A method for correcting presbyopia in a patient, the method comprising:
(a) determining a monovision to be performed on the patient's eyes; and
(b) determining a higher-order aberration to be induced in at least one of the patient's eyes; and
(c) performing the monovision determined in step (a) and for inducing the higher-order aberration determined in step (b).

2. The method of claim 1, wherein the higher-order aberration is induced in only one of the patient's eyes.

3. The method of claim 1, wherein the higher-order aberration is induced in both of the patient's eyes.

4. The method of claim 3, wherein different higher-order aberrations such as secondary and tertiary spherical aberrations are induced in the patent's eyes.

5. The method of claim 3, wherein the same higher-order aberration is induced in both of the patient's eyes.

6. The method of claim 1, wherein the higher-order aberration comprises a spherical aberration.

7. The method of claim 6, wherein the spherical aberration is induced at least in a non- dominant eye.

8. The method of claim 1, wherein step (c) comprises providing optics for performing the monovision and inducing the higher-order aberration.

9. The method of claim 8, wherein the optics comprise intraocular lenses.

10. The method of claim 8, wherein the optics comprise contact lenses.

11. The method of claim 1, wherein step (c) comprises surgery.

12. The method of claim 11, wherein the surgery comprises laser refractive surgery.

13. The method of claim 1, wherein steps (a) and (b) are performed using an adaptive optics vision simulator.

14. A system for providing correction for presbyopia in a patient, the system comprising:
an input for inputting measurements of existing aberrations in the patient's eyes;
a processor in communication with the input, the processor being configured to receive the measurements, to determine a monovision to be performed on the patient's eyes, and to determine a higher-order aberration to be induced in at least one of the patient's eyes; and
an output, in communication with the processor, for outputting a specification for performing the monovision and for inducing the higher-order aberration.

15. The system of claim 14, wherein the output comprises a printer.

16. The system of claim 14, wherein the output comprises a display.

17. The system of claim 14, wherein the output comprises a lens fabricator.

18. The system of claim 14, wherein the processor is configured such that the higher-order aberration is induced in only one of the patient's eyes.

19. The system of claim 14, wherein the processor is configured such that the higher-order aberration is induced in both of the patient's eyes.

20. The system of claim 19, wherein the processor is configured such that different higher-order aberrations are induced in the patent's eyes.

21. The system of claim 19, wherein the processor is configured such that the same higher-order aberration is induced in both of the patient's eyes.

22. The system of claim 14, wherein the processor is configured such that the higher-order aberration comprises a spherical aberration.

23. The system of claim 22, wherein the processor is configured such that the spherical aberration is induced at least in a non-dominant eye.

24. The system of claim 14, wherein the processor is configured such that the monovision and higher-order aberration are carried out by surgery.

25. The system of claim 24, wherein the processor is configured such that the surgery comprises laser refractive surgery.

26. The system of claim 14, wherein the input comprises an adaptive optics vision simulator.

27. Optics for correcting presbyopia in a patient, the optics comprising:
an optical element for the patient's left eye; and
an optical element for the patient's right eye;
wherein the optical elements are configured to perform a monovision on the patient's eyes and to induce a higher-order aberration in at least one of the patient's eyes.

28. The optics of claim 27, wherein the optical elements are configured such that the higher-order aberration is induced in only one of the patient's eyes.

29. The optics of claim 27, wherein the optical elements are configured such that the higher-order aberration is induced in both of the patient's eyes.

30. The optics of claim 29, wherein the optical elements are configured such that different higher-order aberrations are induced in the patent's eyes.

31. The optics of claim 29, wherein the optical elements are configured such that the same higher-order aberration is induced in both of the patient's eyes.

32. The optics of claim 27, wherein the optical elements are configured such that the higher-order aberration comprises a spherical aberration.

33. The optics of claim 32, wherein the optical elements are configured such that spherical aberration is induced at least in a non-dominant eye.

34. The optics of claim 27, wherein the optical elements comprise intraocular lenses.

35. The optics of claim 27, wherein the optical elements comprise contact lenses.
